(19) United States
(12) Patent Application Publication
(10) Pub. No.: US 2018/0153870 A1
(43) Pub. Date: Jun. 7, 2018

(54) BIPERIDEN FOR TREATING CANCER

(71) Applicant: Universitätsklinikum Hamburg-Eppendorf, Hamburg (DE)

(72) Inventors: Alexander T. El Gammal, Hamburg (DE); Jakob R. Izbicki, Hamburg (DE); Leonie Konczalla, Hamburg (DE); Daniel Perez, Hamburg (DE)

(21) Appl. No.: 15/579,313

(22) PCT Filed: Jun. 2, 2016
(86) PCT No.: PCT/EP2016/062444

§ 371 Date: Dec. 4, 2017

(30) Foreign Application Priority Data
Jun. 2, 2015 (DE) 10 2015 210 224.6

(51) Int. Cl. A61K 31/4453; A61K 31/5415; A61P 35/00

(52) U.S. Cl. CPC A61K 31/4453; A61K 2300/00; A61P 35/00; A61K 31/5415

(57) ABSTRACT

The present invention relates to the use of the compound biperiden as a MALT1 inhibitor in the treatment of a cancerous disease. The invention in particular relates to a combination of biperiden and a phenothiazine for use in the treatment of a cancerous disease. In a further aspect, the present invention is directed to a pharmaceutical composition for the treatment of a cancerous disease which comprises biperiden as a MALT1 inhibitor. Furthermore, a pharmaceutical composition is disclosed which comprises biperiden and at least a phenothiazine. In a further aspect, the invention relates to the use of biperiden in the treatment of a cancerous disease. The invention further provides a kit which comprises a container that includes biperiden and at least one container that includes a phenothiazine compound.

The present invention relates to the use of the compound biperiden as a MALT1 (mucosa-associated lymphoid tissue lymphoma translocation protein 1) inhibitor in the treatment of a cancerous disease. The invention in particular relates to a combination of biperiden and a phenothiazine for use in the treatment of a cancerous disease. In a further aspect, the present invention is directed to a pharmaceutical composition for the treatment of a cancerous disease which comprises biperiden as a MALT1 inhibitor. Furthermore, a pharmaceutical composition is disclosed which comprises biperiden and at least a phenothiazine. In a further aspect, the invention relates to the use of biperiden in the treatment of a cancerous disease. The invention further provides a kit which comprises a container that includes biperiden and at least one container that includes a phenothiazine compound.

### BACKGROUND OF THE INVENTION

Neoplastic diseases are characterized by an uncontrolled growth of abnormal cells, wherein these cells can propagate in an uncontrolled way and form metastases in other organs under certain conditions. In the industrial states approximately 20% of all deaths are due to cancerous diseases. The currently used therapeutic concepts are either based on the surgical removal of the neoplastic tissue, radiation or chemotherapy. Chemotherapeutically active substances which are suitable for the treatment of specific neoplastic diseases have been described in the prior art in large numbers. For and overview see reference [1].

However, about half of all neoplastic tissues fail to respond or only insufficiently respond to treatment with the currently available chemotherapeutic drugs. This is particularly true for tumors which are derived from the lung, colon, pancreas, liver or kidney. These tumors are often not susceptible to currently available chemotherapeutic drugs, since they process mechanisms which confer resistance to the substances. Moreover, a number of tumors which are initially susceptible to the employed chemotherapeutic agents lose their susceptibility to an active ingredient during treatment with said active ingredient and become resistant. The result can be that, after several treatment cycles, an active ingredient can no longer inhibit the growth of a tumor. For this reason, it is of great interest to identify new therapeutic active ingredients which can specifically be used also for the treatment of such tumors.

The inhibition of apoptosis is a mechanism that has been particularly well investigated. By this mechanism, tumor cells can prevent their death and induce uncontrolled proliferation. It is assumed that most cells with a cell cycle that is disturbed by oncogenic mutation are normally removed by apoptosis. Caspases and paracaspases play a crucial role during apoptosis. A number of these proteolytic enzymes interact with each other in complex signal transduction pathways to mediate death of the cells in response to outer or inner signals. For example, it has been shown that by inhibition of specific caspases, apoptosis in tumor cells can be induced.

Against this background, it is an objective of the present invention to provide new pharmaceutically active compounds and compositions that allow for an effective treatment of different cancerous diseases. According to the invention, this objective is reached by the compounds and pharmaceutical compositions according to the enclosed claims.

The present invention is based on the surprising insight that the active ingredient biperiden in specific tumors, such as pancreas carcinoma, lung carcinoma or esophagus carcinoma, can inhibit cell proliferation and, moreover, induce apoptosis and cause death to the cells of the tumor. Biperiden has been used for several decades as an agent against Parkinson's disease. Apart from that, biperiden is also used in the antipsychotics therapy for reducing drug related extrapyramidal side effects, such as body rigidity and gaze palsy. The use of biperiden in cancer therapy has not yet been contemplated in the prior art.

In addition, it was found in the present invention that the combined administration of biperiden and a phenothiazine, such as mepazine, results in an improved therapeutic effect which cannot be explained by additive effects.

### DESCRIPTION OF THE INVENTION

The present invention contemplates the use of the active ingredient biperiden for the treatment of a cancerous disease. As found in the course of the invention, the effect of biperiden is based on the inhibition of the MALT1 activity in the tumor cells. Biperiden is an active ingredient from the group of anticholinergics that is widely used against Parkinson's disease and has been well described in the art. The active ingredient biperiden is a racemate of the two enantiomers (1S)-1-[(1 S,2R,4S)-bicyclo[2.2.1]hept-5-en-2-yl]-1-phenyl-3-(1-piperidinyl)-1-propanol (formula Ia; referred to in the course of the invention also as (−)-biperiden) und (1R)-1-[(1R,2S,4R)-Bicyclo[2.2.1]hept-5-en-2-yl]-1-phenyl-3-(1-piperidinyl)-1-propanol (see formula Ib; referred to in the course of the invention also as (+)-biperiden). While biperiden is preferably used as a racemate of (−)-biperiden and (+)-biperiden in the course of the present invention, the isolated use of the respective enantiomers (−)-biperiden and (+)-biperiden for the recited therapeutic application is also encompassed by the invention. Accordingly, the present invention in a first aspect relates to a compound having the formula

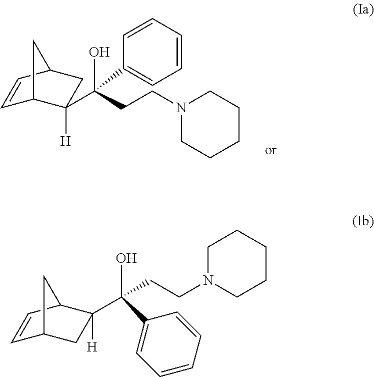 [SMILES: O[C@](CCN1CCCCC1)(c1ccccc1)C1CC2C=CC1C2 ; O[C@@](CCN1CCCCC1)(c1ccccc1)C1CC2C=CC1C2]

or a pharmaceutically acceptable salt or solvate thereof for use as a MALT1 inhibitor in a method of treating a cancerous disease.

It will be evident for a skilled person that the above depicted structures (−)-biperiden and (+)-biperiden can be substituted or otherwise modified at one or several positions, as long as these modifications neither substantially affect the inhibitory effect of biperiden on MALT1 activity nor lead to adverse results in terms of toxicity. For example, one or more hydrogen atoms of the C—H bonds of the heterocyclic ring systems can be substituted by halogen atoms, such as chlorine, bromine or iodine atoms. Further, the hydrogen of the C—H bonds can also be replaced by an alkyl group such as methyl, ethyl or propyl.

Apart from (−)-biperiden and (+)-biperiden, racemate mixtures thereof or substituted derivatives thereof, pharmaceutically acceptable salts of the respective enantiomers can be used as well. The term “pharmaceutically acceptable salt” refers to non-toxic acid addition salts, and alkali metal and alkaline earth metal salts, respectively. Exemplary acid addition salts include hydrochloride, hydrobromide, sulfate, bisulfate, acetate, oxalate, phosphate, citrate, maleate, fumarate, succinate, tartrate and lauryl sulfate. Exemplary alkali metal or alkaline earth metal salts include sodium, potassium, calcium and magnesium salts. In addition, ammonium salts and salts with organic amines can be used as well. Apart from the calcium salt, any other pharmaceutically acceptable cationic salt can be employed. The salts are, for example, salts that are obtained by reaction of the free acid form of biperiden with a suitable base, such as sodium hydroxide, sodium methoxide, sodium hydride, potassium methoxide, magnesium hydroxide, calcium hydroxide, choline, diethanolamine, and others which are known in the prior art. Solvates of (−)-biperiden and (+)-biperiden are also part of the present invention. Solvates occur upon the addition of one or more solvent molecules to an active ingredient compound according to the invention (i.e. biperiden). If the solvent is water, said addition is a hydration. Solvates of the contemplated active ingredient compound can be held together by ionic bonds and/or covalent bonds.

According to the invention, it is preferred that during treatment both enantiomers of biperiden are administered, that is both the compound according to formula (Ia) and the compound of the formula (Ib). Preferably a racemate is used which includes (−)-biperiden and (+)-biperiden in a ratio of about 10:1, 5:1, 2:1, 1:1, 1:2, 1:5, or 1:10. A ratio of (−)-biperiden to (+)-biperiden of about 1:1 is particularly preferred.

The presently contemplated biperiden compound, that is (−)-biperiden, (+)-biperiden, a racemate of both enantiomers, or salts, solvates or derivatives thereof can be used according to the invention for the treatment of a number of cancer diseases. Cancer diseases, that can be effectively treated by a biperiden compound as contemplated herein comprise bronchial carcinoma, colon carcinoma, breast carcinoma, prostate cancer, liver cancer, pancreatic cancer, bladder cancer, skin cancer, ovarian cancer, hematologic cancer diseases, lung cancer, cancer types of the genitourinary system in men and women, cancer of the adrenal cortex (phaeochromocytoma), cancer diseases of the brain, stomach cancer, kidney cancer, uterus cancer, bone cancer, esophagus cancer, Kaposi's sarcoma, oropharyngeal cancer diseases, testicular cancer, thyroid cancer, lymphoma, adrenocortical cancer diseases, gall bladder cancer, multiple myeloma, small intestine cancer, anal cancer, pancreatic cancer, Burkitt lymphoma, bile duct cancer, cervical cancer, urethral cancer, laryngeal cancer, bone cancer, Hodgkin lymphoma, non-Hodgkin lymphoma, Wilms' tumor, plasma cell myeloma or retinoblastoma.

In a preferred embodiment of the present invention the cancer disease to be treated is selected from the group consisting of pancreas carcinoma, lung carcinoma, bronchial carcinoma and/or esophagus carcinoma. According to a particularly preferred embodiment, the cancer disease to be treated is a disease of the pancreas. Pancreatic carcinoma is one of the most aggressive cancer types, and only few patients survive a period of 5 years after diagnosis [2-3]. According to a preferred embodiment, the pancreas carcinoma is a pancreatic adenocarcinoma, such as a pancreatic ductal adenocarcinoma (PDAC), or a neuroendocrine tumor.

Preferably, the cancer disease is a MALT1-dependent disease that is a cancer disease in which the tumor cells express the protease MALT1. Preferably, the cancer disease is a disease in which the tumor cells express MALT1 stronger than the respective non-cancerous cells of the respective tissue. The MALT1 expression in the tumor cells is at least 50% stronger than in the non-cancerous cells, preferably at least 100% stronger, 200% stronger, 300% stronger, 400% stronger or 500% stronger. In a further embodiment the cancer disease is a disease in which MALT1 is activated in the tumor cells continuously or through a signal transduction pathway, while such activation does not occur in the respective non-cancerous cells. The compound of the formula (Ia) and/or the compound of the formula (Ib) is preferably administered in a concentration which is sufficient for effecting an inhibition of the MALT1 protease in the tumor cells.

The biperiden compound contemplated herein and corresponding pharmaceutical compositions, which comprise this biperiden compound can be combined with known chemotherapeutic agents for providing an improved effectiveness. For example, the biperiden compound can be administered with alkylating agents; alkyl sulfonates; aziridines, such as thiotepa; ethylenimine; anti metabolites; folic acid analogues, such as methotrexate (Farmitrexat®, Lantarel®, METEX®, MTX Hexal®); purine analogues, such as azathioprine (Azaiprin®, AZAMEDAC®, Imurek®, Zytrim®), cladribine (Leustatin®), fludarabine phosphate (Fludara®), mercaptopurine (MERCAP®, Puri-Nethol®), pentostatin (Nipent®), thioguanine (Thioguanin-Wellcome®) or fludarabine; pyrimidine analogues, such as cytarabine (Alexan®, ARA-Cell®, Udicil®), fluorouracil, 5-FU (Efudix®, Fluoroblastin®, Ribofluor®), gemcitabine (Gemzar®), doxifluridine, azacitidine, carmofur, 6-azauridine, floxuridin; nitrogen mustard derivatives, such as chlorambucil (Leukeran®), melphalan (Alkeran®), chlornaphazin, estramustine, mechlorethamine; oxazaphosphorines, such as cyclophosphamide (CYCLO-Cell®, Cyclostin®, Endoxan®), ifosfamide (Holoxan®, IFO-Cell®) or trofosfamide (Ixoten®); nitrosourea, such as bendamustine (Ribomustin®), carmustine (Carmubris®), fotemustine (Muphoran®), Iomustine (Cecenu®, Lomeblastin®), carmustine, chlorozotocine, ranimustine or nimustine (ACNU®); hydroxyurea (Litalir®); vinca alkaloids and taxanes, such as vinblastine (Velbe®), Vindesin (Eldisine®), vinorelbine (Navelbine®), docetaxel (Taxotere®), or paclitaxel (Taxol®); platinum compounds, such as cisplatin (Platiblastin®, Platinex®) or carboplatin (Carboplat®, Ribocarbo®); sulfonate esters, such as busulfan (Myleran®), piposul-fan or treosulfan (Ovastat®); anthracyclines, such as doxorubicine (Adriblastin®, DOXO-Cell®), daunorubicine (Daunoblastin®), epirubicine (Farmorubicin®), idarubicine (Zavedos®), amsacrine (Amsidyl®) or mitoxantrone (Novantron®); and with derivatives, tautomers and pharmaceutically active salts of the above recited compounds. The combination of the biperiden compound with anti-angiogenic agents, e.g. with the monoclonal antibody bevacizumab (Avastin®), denosumab (Prolia®, XGEVA®; or with tyrosine kinase inhibitors, such as sorafenib (Nexavar®) or sunitinib (Sutent®), is also possible. The combination of the biperiden compound with therapeutic antibodies, such as trastuzumab (Herceptin®), gemtuzumab (Mylotarg®), panitumumab (Vectibix®) or edrecolomab (Panorex®) are also encompassed according to the invention.

The biperiden compound contemplated herein and the corresponding pharmaceutical compositions which comprise the biperiden compound can moreover be combined with a conventional radiation therapy. The radiation can be applied prior to or after administration of the combination preparation of the invention in accordance with methods known in the art. The radiation can comprise gamma radiation, x-rays and radiation emitted from radioisotopes. The radiation therapy can further comprise particle radiation (electrons, protons, neutrons). Corresponding radiation doses, which are used for the treatment of tumors, are known to the person skilled in the field of radiation therapy. Depending on the type of tumors total doses of e.g. 20-80 Gy can be used.

Particularly preferred effects are achieved upon combination of the biperiden compound with a phenothiazine compound that inhibits the MALT1 protease. It was found in the course of the present invention that the combination of biperiden with such a phenothiazine compound has a much stronger inhibitory effect on the proliferation of cancer cells than expected. It is known in the prior art that phenothiazines, such as mepazine, promazine, and thioridazine can inhibit the proliferation of cancer cells through the inhibition of the MALT1 protease. As can be seen from the below examples, the anti-proliferative effect that was achieved by a combination of biperiden and phenothiazine is significantly stronger than it could have been expected from the addition of the distinct effects. Instead, the effect achieved by the combination was based on a synergistic action of the distinct active ingredients. As used herein, a synergistic effect is to be understood as an effect that is stronger than it could have been expected from the mere addition of the effects observed after administering the components separately. A synergistic effect allows the administration of lower amounts of biperiden or phenothiazine, respectively, such that the tolerability of the composition of the invention is normally improved for the patient. Under certain conditions, the distinct components of the combination, that is the biperiden compound and the phenothiazine compound, can even be administered in sub-therapeutic doses which would not show any effect on the course of the cancer disease to be treated when administered solely.

Therefore, in one aspect of the invention the treatment method comprises, apart from the administration of the biperiden compound, also the administration of a phenothiazine compound which inhibits the MALT1 protease before, simultaneously with or after administration of the biperiden compound. Preferably, the phenothiazine compound which inhibits MALT1 protease is a compound which is selected from the group consisting of mepazine, thioridazine, promazine, and pharmaceutically acceptable salts, derivatives or solvates thereof. Particularly preferred is the use of mepazine all salts, derivatives or solvates thereof in combination with biperiden.

Preferably, the biperiden compound and the phenothiazine compound, such as mepazine, will be present in a single pharmaceutical composition. For the practice of the present invention, it is however not obligatory that both active ingredients are present in a single pharmaceutical composition. Rather, the biperiden compound and the phenothiazine compound can also be present in separate formulations, which are administered to the patient to be treated simultaneously or at different times.

Thus, in a further aspect, the present invention relates to a pharmaceutical composition, comprising:

a) compound having the formula

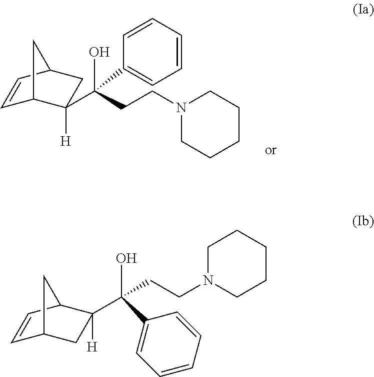 [SMILES: O[C@](CCN1CCCCC1)(c1ccccc1)C1CC2C=CC1C2 ; O[C@@](CCN1CCCCC1)(c1ccccc1)C1CC2C=CC1C2]

or a pharmaceutically acceptable salt or solvate thereof, and
   b) at least one phenothiazine compound.

Such a composition comprises, apart from the biperiden compound, hence at least one phenothiazine compound, such as mepazine or a salt or solvate thereof, as a single formulation, i.e. a single pharmaceutical composition. The active ingredient of the combination can be combined, for example, in vitro, i.e. prior to the administration to a patient to give a single delivery form, provided that none of the two components shows a loss in the MALT1 inhibitory activity when mixing it with the other component of the combination. For example, biperiden and mepazine which are present in a lyophilized mixture can be reconstituted to an infusion solution or injection solution by adding a suitable solvent. Moreover, the pharmaceutical composition can be provided as a single dosage form, for example in the form of a pill for the oral administration.

Alternatively, the biperiden compound in the phenothiazine compound can occur in separate formulations which are administered to the patient simultaneously or at different times. For example, the biperiden compound and the phenothiazine compound can be administered at different days of a treatment cycle. The biperiden compound can, for example, be administered daily within a repeating treatment cycle of one week, while the phenothiazine compound, e.g. mepazine, a salt or solvate thereof, is administered only at a specific day of this cycle. If the biperiden compound and phenothiazine compound are to be administered at different times, it is advisable to provide the active ingredients in separate packaging units (for example in several ampules). In this context, the biperiden compound in the phenothiazine compound can be present in the same or in different delivery forms, as described in more detail below.

In one embodiment of the invention, the biperiden compound is administered to the patient prior to the administration of the phenothiazine. Preferably, the administration of the biperiden compound is administered 1 to 24 hours prior to the administration of the phenothiazine compound. In a preferred embodiment the administration of the biperiden compound occurs 12 to 16 hours prior to the administration of the phenothiazine. In a further preferred embodiment, the phenothiazine compound is administered to the patient prior to the administration of the biperiden compound. Preferably, the administration of the phenothiazine compound occurs 1 to 24 hours prior to the administration of the biperiden compound. In a particularly preferred embodiment, the administration of the phenothiazine compound occurs 12 to 16 hours prior to the administration of the biperiden compound.

In a particularly preferred embodiment the composition according to the invention comprises a biperiden compound as defined above and mepazine or a pharmaceutically acceptable salt, derivative or solvate thereof. In an alternative embodiment, the composition according to the invention comprises a biperiden compound as defined above and thioridazine or a pharmaceutically acceptable salt, derivative or solvate thereof. In yet another alternative embodiment, the composition according to the invention comprises a biperiden compound as defined above and promazine or a pharmaceutically acceptable salt, derivative or solvate thereof.

The compositions according to the invention which comprise the biperiden compound can be administered in any suitable delivery form known in the art. Such methods and suitable excipients and carriers are described, for example, in “Remington: The Science and Practice of Pharmacy”, Lippincott Williams & Wilkins; 21^{st} ed. (2005). Such formulations comprise, for example, compositions for the oral, rectal, nasal or parental (including subcutaneous, intramuscular, intravenous and intradermal) administration. The compositions can occur in the form of granules, powders, tablets, capsules, syrup, suppositories, injection solutions, emulsions or suspensions.

Normally, the compositions of the invention comprise, apart from the biperiden compound in the phenothiazine compound, one or more pharmaceutically acceptable carriers, which are physiologically compatible with the other ingredients of the compositions. The compositions are preparations of the invention which can include, apart from the actual active ingredients, also further excipients, binders, diluents or comparable substances.

For the oral, buccal, and sublingual administration solid formulations such as powders, suspensions, granules, tablets, pills, capsules and gel caps are normally used. These can be prepared, for example, by mixing the active ingredients or their salts with at least one additive or at least one excipient. Such excipients and carriers are disclosed for example in “Remington: The Science and Practice of Pharmacy”, Lippincott Williams & Wilkins; 21^{st}ed. (2005). For example, microcrystalline cellulose, methylcellulose, hydroxypropyl methylcellulose, casein, albumin, mannitol, dextran, sucrose, lactose, sorbitol, starch, agar, alginate, pectins, collagen, glyceride, or gelatine can be used as additives or excipients. Further, oral dosage forms can comprise antioxidants (e.g. ascorbic acid, tocopherol or cysteine), lubricants (e.g. magnesium stearate), preservatives (e.g. paraben or sorbic acid), disintegrants, binders, thickeners, taste enhancers, dyes and similar substances.

Liquid formulations which are suitable for oral administration can occur, for example, as emulsions, syrups, suspensions or solutions. These formulations can be prepared by use of the sterile liquid as a pharmaceutical carrier (e.g. oil, water, alcohol or combinations thereof) in the form of liquid suspensions or solutions. For the oral or parenteral administration, pharmaceutically acceptable surfactants, suspending agents, oils or emulsifiers can be added. Oils which are suitable for being used in liquid dosage forms comprise, for example, olive oil, sesame oil, peanut oil, rape oil, and corn oil. Suitable alcohols comprise ethanol, isopropyl alcohol, hexadecyl alcohol, glycerol and propylene glycol. Suspension can also include fatty acid ester, such as ethyl oleate, isopropyl myristate, fatty acid glycerides, and acetylated fatty acid glycerides. Furthermore, substances like mineral oil or petrolatum are often added to suspensions.

Formulations which are suitable for injections typically comprise aqueous suspensions or oil suspensions which have been prepared by use of the suitable dispersing agent or suspending agent. Injectable forms can occur in solution or in the form of the suspension, which has been prepared with the solvent or diluent. Pharmaceutical carriers can be, amongst others, sterilized water, ringer solution or an isotonic aqueous sodium chloride solution. Alternatively, sterile oils can be used as carriers, which are preferably non-volatile. Dosage forms suitable for injection comprise furthermore powders which can be reconstituted in a solvent. Examples include, amongst others, lyophilized or spray-dried powder. For injection, stabilizers or surfactants can optionally be added to these formulations. The formulations can be administered by injection, such as bolus injection, or by continuous infusion.

The route of administration which is most suitable for the respective therapy and the amount of the biperiden compound (and optionally of the phenothiazine compound) to be administered can be determined by a skilled person using routine methods. Parameters which influence the amount of active ingredients in the pharmaceutical composition to be administered comprise the type and severity of the cancer disease, the age, body weight and sex and the overall state of health of the patient to be treated, the simultaneous administration of other therapeutic agents, and other parameters.

Suitable pharmaceutical compositions for the administration to humans will typically comprise 0.01 mg to 80 mg of the biperiden compound per kilogram body weight of the patient. Preferably, the amount of the biperiden compound is in the range of 1 mg to 20 mg per kilogram body weight of the patient, and even more preferably in the range of 5 mg to 15 mg per kilogram body weight of the patient. 10 mg of the biperiden compound per kilogram body weight are particularly preferred. If the pharmaceutical composition, apart from the biperiden compound, comprises a phenothiazine compound, the amount of the phenothiazine compound in the pharmaceutical composition will typically be between 0.01 mg and 150 mg per kilogram body weight of the patient. Preferably, the amount of the phenothiazine compound in the compositions or preparations of the invention will be in the range of 1 mg to 50 mg per kilogram body weight of the patient, and even more preferred in the range of 5 mg to 25 mg per kilogram body weight of the patient. 15 to 20 mg phenothiazine per kilogram body weight are particularly preferred.

The therapeutic effectiveness of the pharmaceutical compositions and preparations of the inventions can be evaluated by using parameters known in the art. These parameters comprise, amongst others, the effectiveness of the composition according to the invention in eradicating tumors, the response rate, the time until progression of the disease and the survival rate of the treated patients. An effect which is directed to a tumor can manifest itself in an inhibition of tumor growth, a delay in tumor growth, the reduction of the tumor, a reduction of the number of tumor cells, a prolongation of the time until the onset of regrowth of the tumor and the delay in the progression of the disease.

Preferably, the compositions and preparations of the invention resulted in a complete response in the patient. As used herein, complete response means the elimination of all clinically detectable disease symptoms as well as the restoration of normal results in blood count, x-ray examination, CT pictures, and the like. Such a response lasts preferably a month after the treatment has been stopped. The anti-proliferative compositions and preparations of the invention can also result in a partial response in the patient. In a partial response the measurable tumor burden in the patient is reduced. Specifically, this means that the number of the tumor cells present in the patient is reduced and no new lesions can be observed. At the same time, an improvement occurs in one or more symptoms which are caused by the disease (e.g. fever, loss of weight, vomiting).

In a still further aspect, the invention relates to the use of a compound of formula (Ia) or (Ib) or pharmaceutically acceptable salt or solvate thereof as a MALT1 inhibitor in the treatment of a cancer disease. Preferably, the compound is used, as described above, for the treatment of pancreatic carcinomas, lung carcinomas, bronchial carcinomas and/or esophagus carcinomas. The use in the treatment of a pancreatic carcinoma is particularly preferred. The use of the compound of formula (Ia) or (Ib) or a pharmaceutically acceptable salt or solvate thereof as a MALT1 inhibitor can further comprise also the administration of a phenothiazine compound. Preferably, the phenothiazine compound is selected from the group consisting of mepazine, thioridazine, promazine and pharmaceutically acceptable salts, derivatives or solvates thereof.

Finally, the invention relates to a kit that comprises the following components:
a) a container comprising a compound of the formula

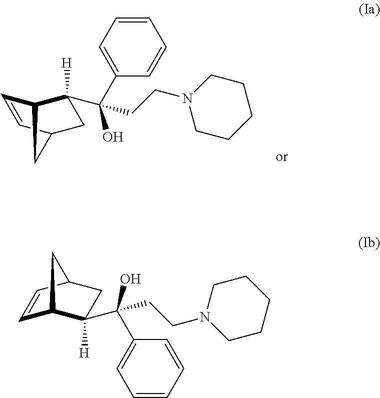

or a pharmaceutically acceptable salt or solvate thereof, and
b) at least one container comprising a phenothiazine compound.

The kit can further include instructions for the combined use of the biperiden compound and a phenothiazine compound in the treatment of a patient with a cancer disease. The instructions can comprise, for example specific amounts and treatment regimens, and they can comprise information concerning the length of administration. The kit can moreover comprise other components which are useful for carrying out the present invention, for example, solvents, excipients, binders, diluents, or similar substances. Furthermore, the kit may also comprise injection syringes, cannulas, catheters and other auxiliary means which are suitable for the administration of the pharmaceutical compositions of the invention.

### DESCRIPTION OF THE FIGURES

FIG. 1 shows the results of in vitro assays for the determination of proliferation of pancreatic cell lines which were treated with different concentrations of biperiden.

FIG. 2 shows the results of in vitro assays for the determination of proliferation of pancreatic cell lines which were treated with different concentrations of mepazine.

FIGS. 3 and 4 show the results of in vitro assays for the determination of proliferation of pancreatic cell lines which were treated with different concentrations of biperiden and mepazine.

FIG. 5 shows the determination of the apoptosis rate in the pancreatic cell lines L3.6pl res, L3.6pl wt, Panc-1, Panc-2, BxPC3 as well as in the reference line HPDE upon treatment of the cells with 29.6 μg/ml biperiden or 25 μM mepazine or a combination of 15 μM mepazine and 3.7 μg/ml biperiden. (*) refers to a trend (p<0.1), * refers to a statistical significance (p≤0.05), ** refers to a high statistical significance (p<0.001).

FIG. 6 shows the results of in vitro assays for the determination of proliferation of human esophageal carcinoma cell lines which were treated with different concentrations of biperiden.

FIG. 7 shows the results of in vitro assays for the determination of proliferation of human esophageal carcinoma cell lines which were treated with different concentrations of mepazine.

FIG. 8 shows the results of in vitro assays for the determination of proliferation of human esophageal carcinoma cell lines which were treated with different concentrations of biperiden and mepazine.

FIG. 9 shows the results of in vitro assays for the determination of proliferation of human bronchial carcinoma cell lines which were treated with different concentrations of biperiden.

FIG. 10 shows the results of in vitro assays for the determination of proliferation of human bronchial carcinoma cell lines which were treated with different concentrations of mepazine.

FIG. 11 shows the results of in vitro assays for the determination of proliferation of human bronchial carcinoma cell lines which were treated with different concentrations of biperiden and mepazine.

### EXAMPLES

The present invention will be described in the following by preferred embodiments which illustrate the invention but should by no means limit the invention.

### Example 1: MTT Proliferation Assays

The cell lines used for the proliferation assays were cultured in different media. The cell lines Pan-1, Panc-2 and BxPC3, which have been derived from the human ductal pancreatic adenocarcinoma (PDAC), were cultured in Dulbecco's modified Eagle medium (DMEM) (Sigma), that was supplemented with 1% penicillin/streptomycin (Life technologies/GIBCO 15140-122) and 10% fetal bovine serum (Life technologies/GIBCO 100500-064). The cell line L3.6pl was cultured in RPMI 1640 medium (Life technologies/GIBCO 72400-21) that was supplemented with 1% penicillin/streptomycin and 10% fetal bovine serum. A sub-clone of the cell line L3.6pl, that was designated L3.6pl res, was resistant to gemcitabine and was also cultured in RPMI 1640 medium (Life technologies/GIBCO 72400-21) that was supplemented with 1% penicillin/streptomycin, 10% fetal bovine serum and 2 μM gemcitabine (GEMZAR, Lily). The human immortalized, non-malignant cell line of the ductal pancreatic epithelium (HPDE) was cultured in keratinocyte-SFM (Life technologies/GIBCO) that was supplemented with 10% fetal bovine serum, 1% penicillin/streptomycin and 1% epidermal growth factor (EGF).

The proliferation rates of the cell lines were determined after stimulation with (i) biperiden, (ii) mepazine, and (iii) stimulation with both active ingredients. For this purpose, cells were seeded in plates with 96 wells at 5000 cells per well and incubated overnight at 37° C. and 5% CO₂. Next morning, the blanket values were measured at 490 nm in an ELISA reader (FLUOstar Omega, BMG LABTECH) after the addition of 100 μL medium and 20 μL MTT substrate (CellTiter 96 AQueous One Solution Cell Proliferation Assay, Promega). Biperiden was added in increasing concentrations (3.71 μg/ml, 11.1 μg/ml und 29.6 μg/ml) in the form of solutions of biperiden hydrochloride (B5311 SIGMA-Aldrich, USA) and DMSO. Mepazine was added in increasing concentrations (15 μM and 25 μM) in the form of solutions of mepazine hydrochloride (MALT1 Inhibitor II, Calbiochem Merck, Millipore Billerica, USA). Combinations of both mepazine and biperiden were added in the following concentrations: 15 μM mepazine+3.71 μg/ml biperiden; 25 μM mepazine+3.71 μg/ml biperiden. The ex-tinction was measured every 24 hours during a period of 72 hours.

The normal distribution of the results was analyzed by Kolgomorov-Smirnov test and Shapiro-Wilk test. Non-parametric variables were analyzed using the Mann-Whitney-U test. If the group was smaller than n=5, Fisher's exact test was used. Normally distributed variables were examined using Student's t-test. The survival analysis was done using the log-rank test and Kaplan-Meier estimations. Spearman's rho test was used for measuring the statistical dependency between two variables. The values are cited as median and interquartile range (IQR). All statistical tests were carried out with SPSS (version 21, IBM).

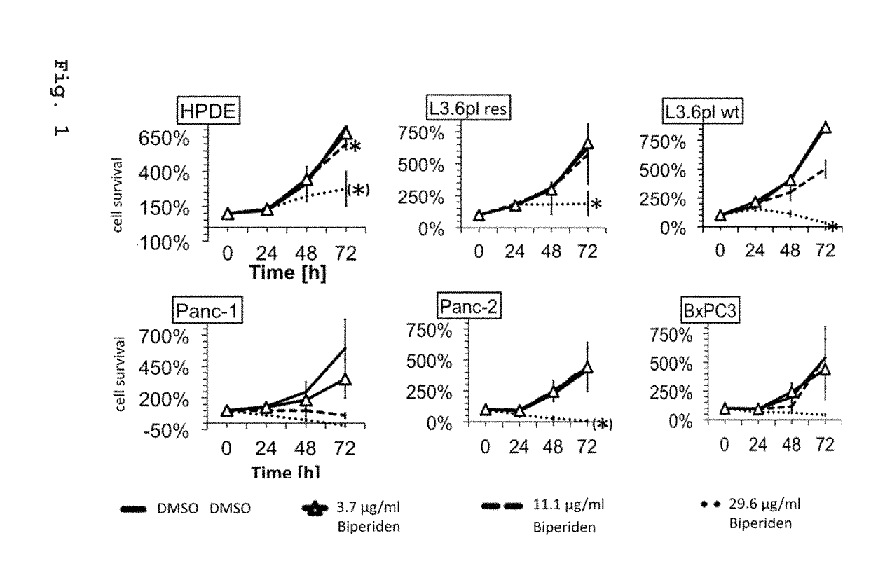

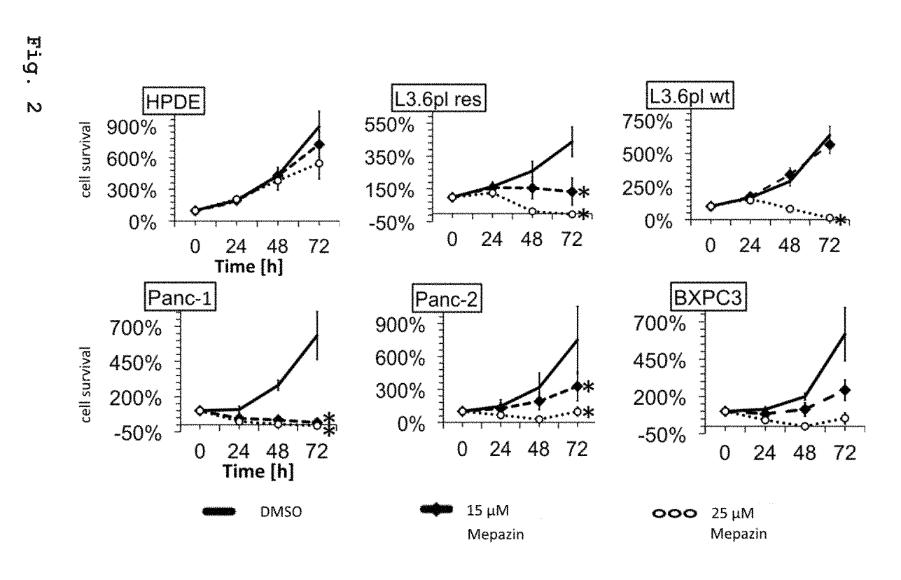

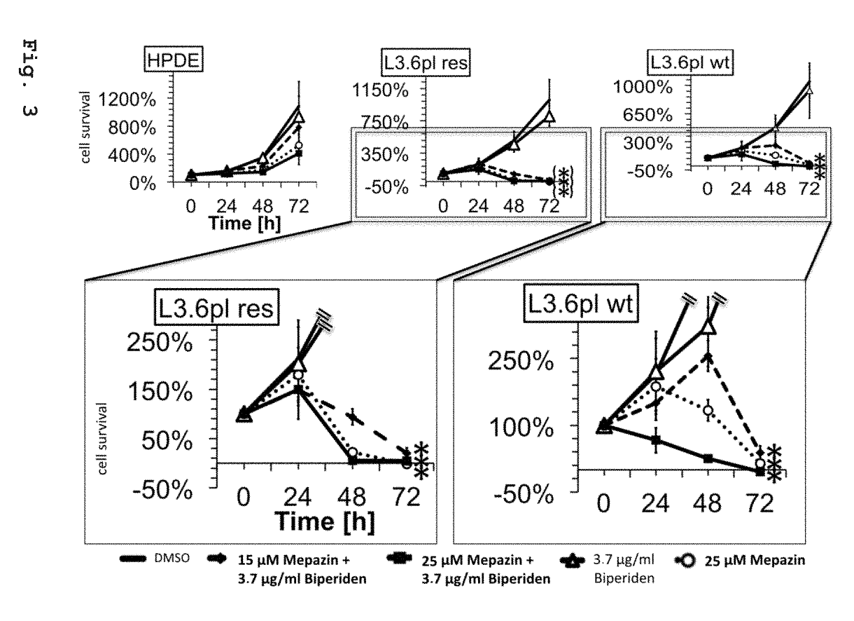

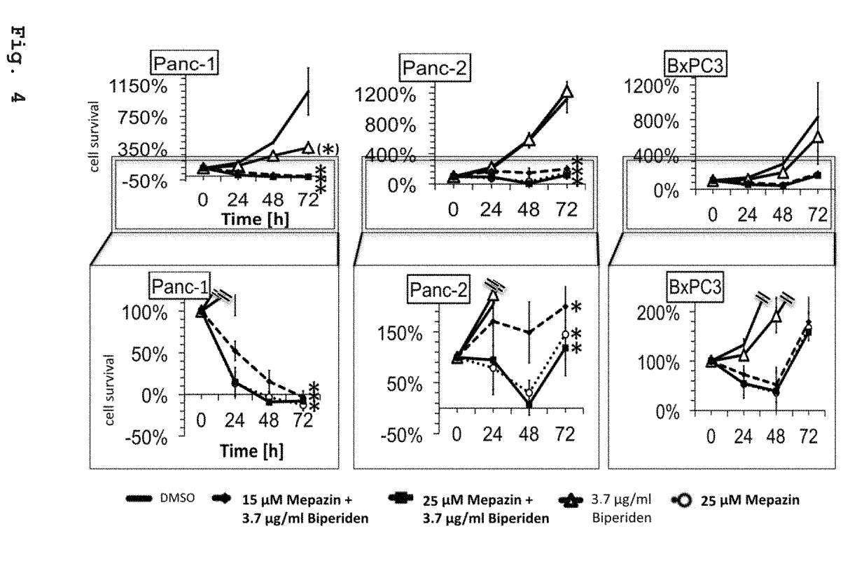

The results of the examination are shown in FIGS. 1 to 4. It can be seen that mepazine and biperiden exert strong inhibitory effects on the proliferation of pancreatic cancer cells. In the cell line HPDE that was treated with 11.1 μg/ml biperiden, the proliferation was reduced slightly. HPDE however showed no statistically significant reaction to mepazine, other doses of biperiden or a combination of mepazine and biperiden.

In contrast, all pancreatic cancer cell lines showed a massive reduction in the proliferation rate in the presence of biperiden, mepazine or a combination of both agents (see FIGS. 1-4). L3.6pl res reduced the proliferation rate by 71.6% in the presence of 29.6 μg/ml biperiden (p=0.022); by 69.4% in the presence of 15 μM mepazine (p=0.031), and the proliferation completely stopped at the doses of 25 μM mepazine (p=0.002). L3.6pl res further showed the trend for a proliferation rate that was reduced by 98% for a combination of 15 μM mepazine and 3.71 μg/ml biperiden (p=0.06) and by 99.5% for 25 μM mepazine and 3.71 μg/ml biperiden (p=0.058).

L3.6pl wt showed a reduction in the proliferation rate by 96.4% in the presence of 29.7 μg/ml biperiden (p=0.02), a reduction by 97.9% for 25 μM mepazine (p=0.004), a reduction by 96.4% at 15 μM mepazine+3.71 μg/ml biperiden (p=0.032), and the proliferation was completely stopped in the presence of 25 μM mepazine+3.71 μg/ml biperiden (p=0.03).

Panc-1 showed a statistically significant reduction by 97.7% in the presence of 15 μM mepazine (p=0.005), and the proliferation was completely stopped in the presence of 25 μM mepazine (p=0.004), 15 μM mepazine+3.71 μg/ml biperiden (p=0.023), and 25 μM mepazine+3.71 μg/ml biperiden (p=0.022).

Panc-2 showed a trend for a proliferation rate that was reduced by 98.2% at 29.6 μg/ml biperiden (p=0.077), a statistically significant reduction by 56.1% in the presence of 15 μM mepazine (p=0.008), a reduction by 86.9% at 25 μM mepazine (p=0.001), a reduction by 82.2% at 15 μM mepazine+3.71 μg/ml biperiden (p=0.031), and a reduction by 89.5% in the presence of 25 μM mepazine+3.71 μg/ml biperiden (p=0.006).

BxPC3 visibly reduced the proliferation rates, wherein the reduction was however not in a statistically significant range.

### Example 2: Determination of Apoptosis

To determine the effect of biperiden, mepazine and combinations of the two active ingredients on apoptosis in cell lines, a “cleaved caspase 3” sandwich ELISA was used. The pancreatic cancer cell lines L3.6pl res, L3.6pl wt, Panc-1, Panc-2 and the reference cell line HPDE were treated with 29.6 μg/ml biperiden or 25 μM mepazine or 15 μM mepazine+3.7 μg/ml biperiden or only with vehicle (DMSO). The cleaved caspase 3 was measured 24, 48 and 72 hours after incubation. The apoptosis within the cells was measured using a PathScan “cleaved caspase 3” sandwich ELISA (Cell Signaling, #7190C) according to the manufacturers instructions. Each experiment was carried out at least 3 times in duplicate.

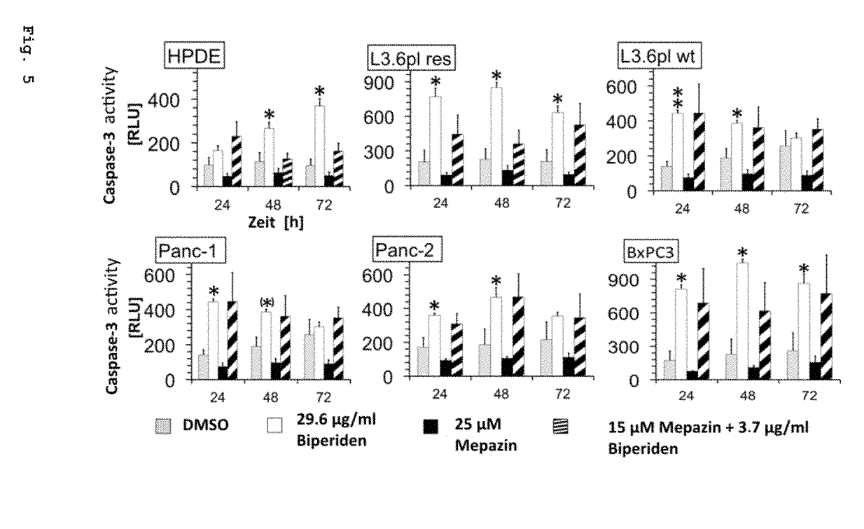

The results are shown in FIG. 5. In none of the tested cell lines, apoptosis was increased after treatment with 5 μM mepazine or 15 μM mepazine+3.7 μg/ml biperiden. In contrast, increased apoptosis was observed in all cell lines after treatment with 29.6 μg/ml biperiden. The apoptosis rate in the cell line HPDE was increased upon treatment with 29.6 μg/ml biperiden by 132% after 48 hours compared to cells which have only been treated with vehicle (265.3±27.8 RLU vs. 114.3±40.8 RLU; p=0.037, t-Test), and by 283.9% after 72 hours (368.4±33.5 RLU vs. 96.0±28.9 RLU; p=0.002, t-test). The apoptosis rate in the cell line L3.6pl res was increased upon treatment with 29.6 μg/ml biperiden by 274% after 24 hours compared to cells which have only been treated with vehicle (770.0±72.5 RLU vs. 205.9±97.8 RLU; p=0.008, t-test), by 273.8% after 48 hours (848.1±45.7 RLU vs. 226.9±91.1 RLU; p=0.003, t-test), and by 204.0% after 72 hours (633.3±55.7 RLU vs. 208.3±101.1; p=0.021, t-test). The apoptosis rate in the cell line L3.6pl wt was increased upon treatment with 29.6 μg/ml biperiden by 210.4% after 24 hours (443.4±18.1 RLU vs. 139.7±29.0 RLU; p<0.001, t-test), and by 83.5% after 48 hours (386.2±15.9 RLU vs. 210.4±53.7 RLU; p=0.028, t-test). The apoptosis rate in the cell line Panc-1 was increased upon treatment with 29.6 μg/ml biperiden by 125.3% after 24 hours (325.1±48.3 RLU vs. 144.2±48.2 RLU; p=0.049). The apoptosis rate in the cell line Panc-2 was increased upon treatment with 29.6 μg/ml biperiden by 109.1% after 24 hours (360.0±11.0 RLU vs. 172.1±49.2 RLU; p=0.029), and by 65.3% after 48 hours (306.0±74.8 RLU vs. 185.2±81.5 RLU; p=0.047, t-test). The apoptosis rate in the cell line BxPC3 was increased upon treatment with 29.6 μg/ml biperiden by 369.8% after 24 hours (813.1±37.7 RLU vs. 173.1±83.7 RLU; p=0.002, t-test), and by 357.5% after 48 hours (1046.1+34.7 RLU vs. 228.7±136.1 RLU; p=0.004, t-test), and by 230.9% after 72 hours (862.8+85.2 RLU vs. 260.8±161.6 RLU; p=0.032).

### Example 3: Determination of the MALT1 Activity and Tumor Cells

The MALT1 activity was measured in the pancreatic cancer cells Panc-1, Panc-2 and in the reference line HPDE as well as in the PMA-stimulated and non-stimulated Jurkat cells. In order to examine whether mepazine, biperiden or a combination of both active ingredients influence the MALT1 activity and pancreatic cancer cells, an assay was performed as described in Nagel et al. [4]. For this purpose, the pancreatic cancer cell lines and the reference cell lines were treated with biperiden (29.6 μg/ml), mepazine (25 μM), a combination of both active ingredients (15 μM mepazine+3.71 μg/ml biperiden) or only with DMSO. The cells were subsequently lysed and precipitated with mouse anti-MALT1 antibody (SC-76677, Santa Cruz, Calif., USA) as previously described by Gungor et al. [5]. As described in the protocol, the fluorogenic substrate AC-LRSR-AMC, which was derived from the C-terminal BCL 10 cleavage site, was used as a substrate for MALT1. The cleavage activity of MALT1 was subsequently determined as relative fluorescence units by a microtiter plate reader (FLUOstar, Omega, BMG Labtech).

The results were determined after 24 hours of incubation with mepazine, biperiden, or a combination of both active ingredients. It was shown that pancreatic cancer cell lines as well as non-malignant, immortalized HPDE cell lines had constitutive MALT1 activity, although to a different extent. As a reference, a non-stimulated Jurkat cell line and a Jurkat cell line that was activated with PMA were examined. Surprisingly, the pancreatic cancer cells showed a higher constitutive MALT1 activity compared to the Jurkat cells.

In the reference cell line HPDE, the incubation with 25 μM mepazine did not influence the MALT1 activity. In contrast, the addition of 29.7 μg/ml biperiden reduced the MALT1 activity 10, 30 and 60 minutes after incubation with the MALT1 substrate significantly (p=0.002; p=0.005; p=0.012; t-test).

In the cell line Panc-1 that was treated with 25 μM mepazine, the MALT1 activity was reduced immediately after addition of the MALT1 substrate (p=0.004; T-test) and also 10, 30, 60 and 90 minutes thereafter (p=0.004; p=0.002; p=0.001; p=0.003; p=0.011; t-test). The same effect was observed upon treatment with a combination of both active ingredients, where 15 μM mepazine+3.71 μg/ml biperiden were added to the cells (p=0.006; p=0.004; p=0.003; P=0.004; p=0.010; t-test). The treatment of the cells with 29.7 μg/ml biperiden visibly reduced the MALT1 activity; however, this effect was not statistically significant.

In the cell line Panc-2, the MALT1 activity was reduced upon treatment with 29.7 μM biperiden immediately after the addition of the MALT1 substrate and also 10 minutes after addition of the MALT1 substrate to a statistically significant extent (p=0.011; p=0.031; t-test). 25 μM mepazine and a combination of both active ingredients, 15 μM mepazine+3.71 μg/ml biperiden, reduced the MALT1 activity, whereas this reduction however was not statistically significant.

### Example 4: Mouse Xenograft Model

For the mouse xenograft model pfp−/−/rag2−/− double knockout mice were used. This mouse model shows a severe disturbance of the NK cell function due to an inactivated pfg gene. Owing to the inactivated rag2 gene, the mouse lacks mature T or B lymphocytes [6-7]. The mouse model was developed by the Taconic Institute (Quality Laboratory Animals and Services for Research, DK8680 Ry, Taconic Europe, Denmark) by crossing the PFPN12 mouse model with the RAGN12 mouse model. This model was back crossed over 12 generations (N12) with C57BL/6NTac, and the colony was maintained by homozygous pairing.

The mouse model was used in the course of the present invention with PDAC cells of the cell line Panc-1, which were administered subcutaneously. 12 days after subcutaneous injection of 10⁶ human Panc-1 tumor cells, the mice were treated daily either with 16 mg/kg mepazine i.p. (n=10), 10 mg/kg biperiden (n=10) i.p. or with a combination of both active ingredients, that is with 16 mg/kg mepazine and 10 mg/kg biperiden (n=10) i.p. The control group (n=10) was not treated.

The daily treatment was performed under identical, standardized conditions. The same 3-days-cycle was used over the complete period of the study: day one comprised the injection of the medication and the determination of the bodyweight by weighing. Day 2 comprised the injection of the medication and the determination of the subcutaneous tumor growth with a caliper. Day 3 comprised the injection of the medication and neuroscoring. The mice showed good compatibility with respect to the administration of biperiden. Overall, mice that had been treated with biperiden as well as mice that had received a combination of both preparations appeared motorically more active compared to mice that received mepazine or no medication (control), respectively.

As soon as the tumors in the control group reached a diameter of approximately 10 mm, the mice from the groups that were treated with biperiden, mepazine or biperiden+mepazine were killed and examined.

After euthanizing, the subcutaneous tumors were removed from the mice. Prior to the surgery, the body of the mouse was disinfected with ethanol. The skin above the tumor was carefully incised, and the tumor was surgically removed from the adjacent tissue. After removal, the tumor was weighed and measured.

For evaluation only mice were used which had developed a tumor. The results showed that the size of the subcutaneous tumor was significantly reduced in mice that were treated with mepazine and biperiden. The volumes of the subcutaneous tumors were measured with a caliper after removal and calculated using the formula length× width² as described in [8]. The tumor sizes of the groups that were treated with biperiden, mepazine or mepazine+biperiden, respectively, were visibly smaller compared to the control groups. The average tumor size in the control group was 729.33 mm³±186.25 mm³ compared to 130.43 mm³±63.96 mm³ in the group that was treated with mepazine (p=0.002; one-sided Fisher's exact test). Upon treatment with both active ingredients, the tumor size was 164.50 mm³±45.25 mm³ compared to 1000.33 mm³±816.86 mm³ in the control group (not significant).

### Example 5: Proliferation Assay

In order to examine whether the effects observed with the human ductal pancreatic adenocarcinoma (PDAC) cell lines also occur with other cancer cell lines, the MTT proliferation assay performed in Example 1 was repeated with three human esophageal carcinoma cell lines and two human lung carcinoma cell lines. These three esophageal carcinoma cell lines OE19, OE33 und KYSE 140 were each cultured in RPMI 1640 medium (Life technologies/GIBCO 72400-21), that was supplemented with 1% penicillin/streptomycin and 10% fetal bovine serum. The cell lines OE19 und OE33 are cells from an adenocarcinoma of the esophagus. KYSE-140 are squamous-cell carcinoma cells of the esophagus.

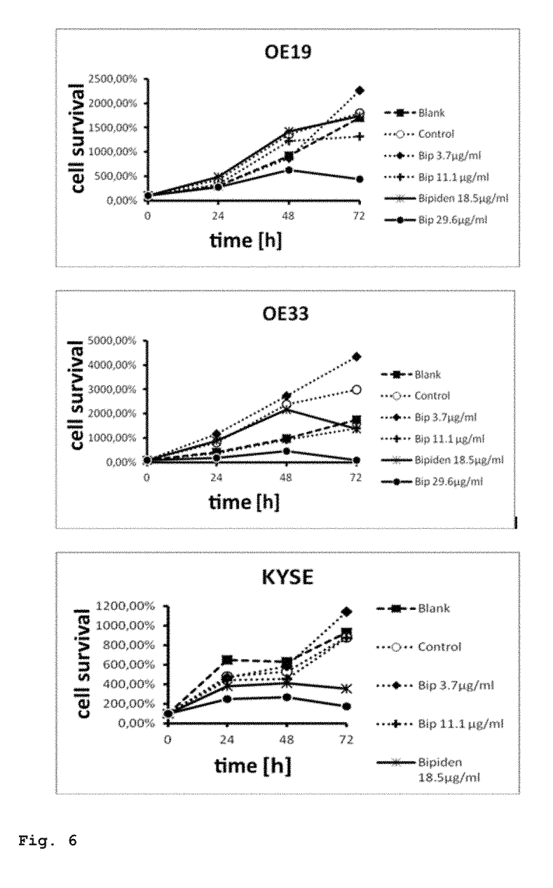

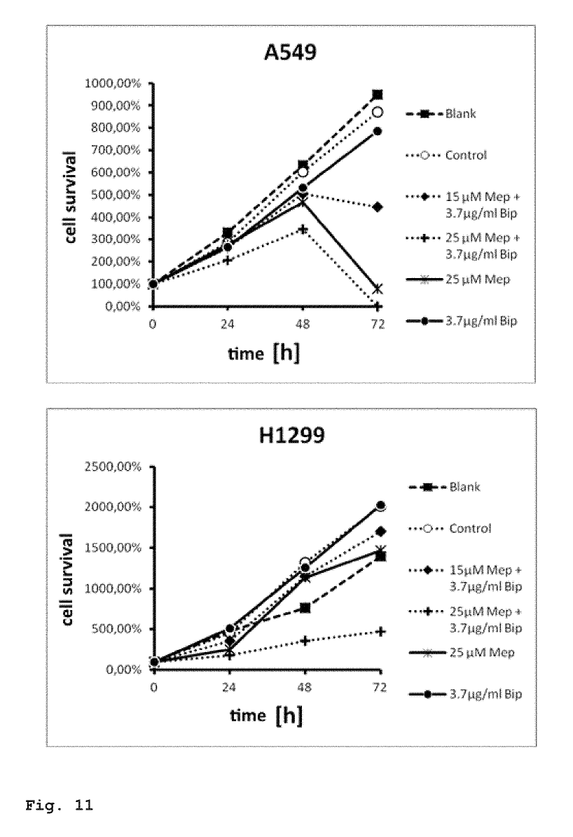

The cell lines from the lung, A549 und H1299, are adenocarcinoma cells of the alveolar basal lamina and cells of a non-small cell bronchial carcinoma, respectively. A549 was cultured in Dulbecco's modified Eagle medium (DMEM) (Sigma), that was supplemented with 1% penicillin/streptomycin (Life technologies/GIBCO 15140-122) and 10% fetal bovine serum (Life technologies/GIBCO 100500-064). H1299 was cultured in RPMI 1640 medium (Life technologies/GIBCO 72400-21) that was supplemented with 1% penicillin/streptomycin and 10% fetal bovine serum. The proliferation assay was carried out as described in Example 1. The results of the assays are shown in FIGS. 6-11.

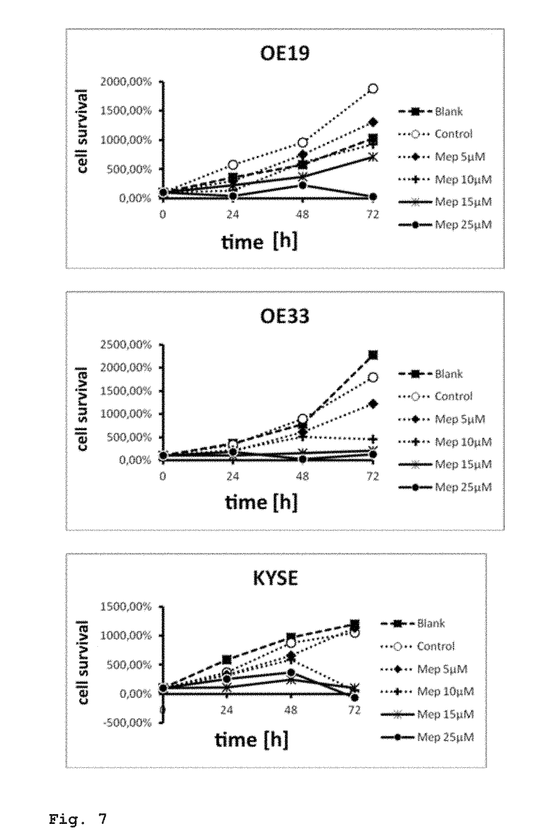

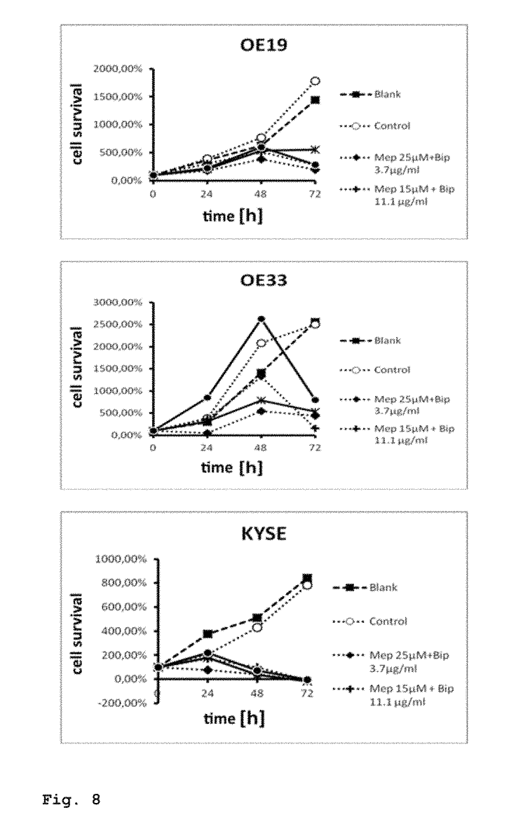

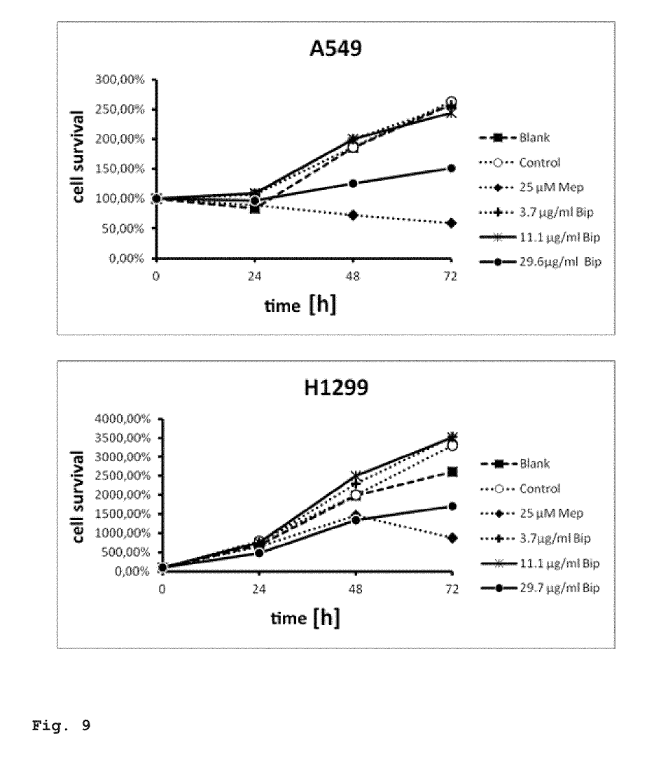

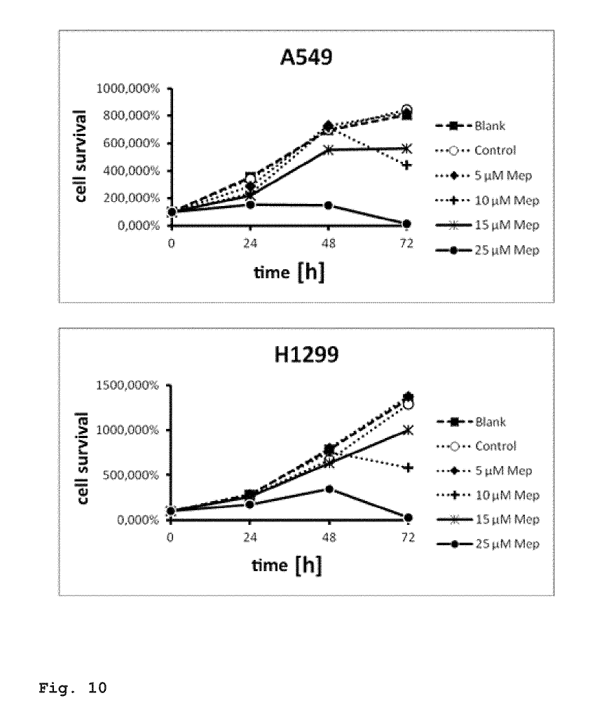

As shown in the figures, mepazine and biperiden inhibited proliferation of esophageal carcinoma cell lines significantly (see FIGS. 6 and 7). The combination of both active ingredients was shown to be particularly effective (see FIG. 8). With the lung carcinoma cell lines, both mepazine and biperiden inhibited proliferation when used separately (see FIGS. 9 and 10). Also with these cell lines, the combined use of both active ingredients was particularly effective (see FIG. 8).

Proliferation assays with additional esophageal carcinoma and bronchial carcinoma cell lines were performed, and substantially the same results were achieved. This shows that the inhibitory effect on proliferation is evidently not restricted to certain cancer types.

### REFERENCES

[1] De Vita, Hellman & Rosenberg, Cancer: Principles and Practice of Oncology, 7. Aufiage 2004, Lipincott, Williams & Wilkins.
[2] Ferrone et al. (2008), J Gastrointest Surg, 12(4): 701-6.
[3] Valle et al. (2014), J Clin Oncol, 32(6): 504-12.
[4] Nagel & Krappmann (2013) Measurement of Endogenous MALT1 Activity. 3. Quelle: http://www.bio-protocol.org/e821.
[5] Gungor et al. (2011), Cancer Res, 71(14): 5009-19.
[6] Shinkai et al. (1992), Cell, 68(5): 855-67.
[7] Walsh et al (1994), Proc Natl Acad Sci USA, 91(23): 10854-8.
[8] Tomayko et al (1989), Cancer Chemother Pharmacol, 24(3): 148-54.

1. Compound having the formula 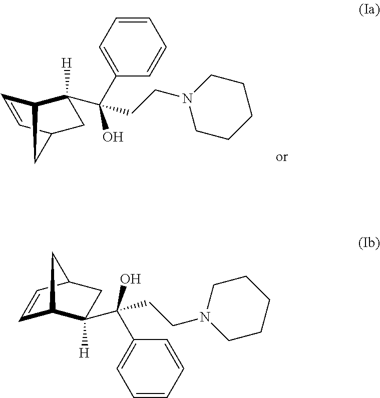
or a pharmaceutically acceptable salt or solvate thereof for use as a MALT1 inhibitor in a method of treating a cancer disease.

2. Compound for use in the method of claim 1, wherein the method comprises both the administration of the compound of formula (Ia) and the compound of formula (Ib).

3. Compound for use in the method of claim 1, wherein the cancer disease is selected from the group consisting of pancreatic carcinoma, lung carcinoma, bronchial carcinoma and/or esophageal carcinoma.

4. Compound for use in the method of claim 3, wherein the cancer disease is a pancreatic carcinoma.

5. Compound for use in the method of claim 1, wherein the method further comprises the administration of the phenothiazine compound.

6. Compound for use in the method of claim 5, wherein the phenothiazine compound is selected from the group consisting of mepazine, thioridazine, promazine and pharmaceutically acceptable salts, derivatives or solvates thereof.

7. Compound for use in the method of claim 1, wherein the compound of formula (Ia) and/or the compound of formula (Ib) is/are added in a concentration that is sufficient for inhibiting the MALT1 protease in the tumor cells.

8. Pharmaceutical composition, comprising:
a) a compound having the formula 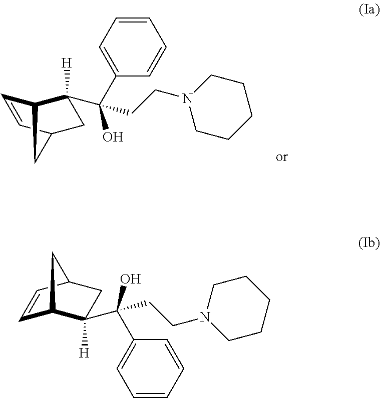
or a pharmaceutically acceptable salt or solvate thereof, and
b) at least one phenothiazine compound.

9. Pharmaceutical composition of claim 8, further comprising a pharmaceutically acceptable carrier or a pharmaceutically acceptable excipient.

10. Pharmaceutical composition of claim 8, wherein the phenothiazine compound is selected from the group consisting of mepazine, thioridazine, promazine and pharmaceutically acceptable salts, derivatives or solvates thereof.

11. Pharmaceutical composition of claim 8, comprising both the compound of formula (Ia) and the compound of formula (Ib).

12. Pharmaceutical composition of claim 8 for use in a method of treating a cancer disease.

13. Pharmaceutical composition for use in a method of claim 12, wherein the cancer disease is selected from the group consisting of pancreatic carcinoma, lung carcinoma, bronchial carcinoma and/or esophageal carcinoma.

14. Pharmaceutical composition for use in a method of claim 13, wherein the cancer disease is a pancreatic carcinoma.

15. Use of a compound according to claim 1 for use as a MALT1 inhibitor in the treatment of a cancer disease.

16. Use of claim 15, wherein the cancer disease is selected from the group consisting of pancreatic carcinoma, lung carcinoma, bronchial carcinoma and/or esophageal carcinoma.

17. Use of claim 16, wherein the cancer disease is a pancreatic carcinoma.

18. Use of claim 15, wherein in the treatment further comprises the administration of the phenothiazine compound.

19. Use of claim 18, wherein the phenothiazine compound is selected from the group consisting of mepazine, thioridazine, promazine and pharmaceutically acceptable salts, derivatives or solvates thereof.

20. Kit, comprising
a) a container comprising a compound having the formula 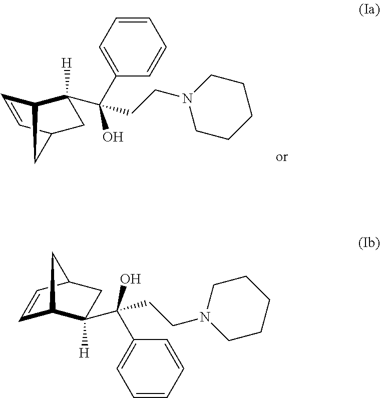
or a pharmaceutically acceptable salt or solvate thereof, and
b) at least one container that comprises a phenothiazine compound.
